(19) United States
(12) Patent Application Publication
(10) Pub. No.: US 2024/0307688 A1
(43) Pub. Date: Sep. 19, 2024

(54) BIMODAL VAGUS NERVE STIMULATION TO TREAT NEURODEGENERATIVE DISORDERS

(71) Applicant: SetPoint Medical Corporation, Valencia, CA (US)

(72) Inventor: Jacob A. LEVINE, West Hempstead, NY (US)

(21) Appl. No.: 18/605,809

(22) Filed: Mar. 14, 2024

Related U.S. Application Data
(60) Provisional application No. 63/490,239, filed on Mar. 14, 2023.
(60) Provisional application No. 63/491,042, filed on Mar. 17, 2023.

(51) Int. Cl. A61N 1/36

(52) U.S. Cl. CPC A61N 1/36103; A61N 1/36053; A61N 1/36157; A61N 1/36171

(57) ABSTRACT

Systems, devices, and methods for using electrical stimulation to treat demyelination disorders and/or disorder of the blood brain barrier are described. For example, described herein are methods of preventing or reducing demyelination by applying a low frequency (e.g., <20 Hz) stimulation concurrently with a higher frequency (e.g., >20 Hz) stimulation that may be paired with a motor task, in order to reduce and/or reverse demyelination.

### CROSS REFERENCE TO RELATED APPLICATIONS

This patent application claims priority to U.S. Provisional Patent Application No. 63/490,239, titled “VAGUS NERVE STIMULATION TO TREAT NEURODEGENERATIVE DISORDERS,” filed on Mar. 14, 2023, and to U.S. Provisional Patent Application No. 63/491,042, titled “VAGUS NERVE STIMULATION TO TREAT NEURODEGENERATIVE DISORDERS,” filed on Mar. 17, 2023, each of which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

A variety of central nervous system (CNS) demyelinating disorders, including multiple sclerosis, acute disseminated encephalomyelitis and neuromyelitis optica spectrum disorders, are difficult to effectively treat. For example, multiple sclerosis (MS) is a neurodegenerative and neuroinflammatory disease characterized by demyelination of nerves in the central nervous system. Although the root cause of demyelination is not well understood, it generally is associated with the formation of lesions on the myelin sheaths and inflammation. Currently, there is no known cure for MS. Current treatments, with modest success, are primarily directed to treating acute attacks and reducing the frequency of attacks in the relapsing-remitting subtype of the disease or treating the symptoms. However, current therapies at best only slow the progression of the disease, and no therapy to date has demonstrated an ability to remyelinate nerves.

Therefore, it would be desirable to provide additional treatment methods and systems that can be used independently or in conjunction with other therapies to reduce the rate or amount of demyelination. Furthermore, it would desirable to provide a therapy that remyelinates nerves and reverses the progression demyelination.

### SUMMARY OF THE DISCLOSURE

Described herein are apparatuses (e.g., devices, systems, including software, hardware and firmware) and methods for applying neural stimulation to treat neurodegenerative and neuroinflammatory disorders, and more specifically apparatuses and methods for applying a specific nerve stimulation to reduce demyelination (e.g., by preventing the immune cell infiltration into the CNS) and, surprisingly, to promote remyelination and/or reverse demyelination in order to treat various neurodegenerative and neuroinflammatory disorders such as, but not limited to, multiple sclerosis. Other conditions that may be treated may include: ALS, myasthenia gravis, spinal cord injury, stroke, traumatic brain injury, Parkinson's, and epilepsy.

In general, these methods and apparatuses (e.g., systems for performing these methods) may include bimodal stimulation. For example, these methods may include the simultaneous (e.g., overlapping and/or concurrent or sequential) use of electrical stimulation of a target nerve or nerves at two separate frequency ranges, a low frequency range (e.g., centered around 10 Hz, between 1 Hz and 29 Hz, between 2 Hz and 25 Hz, between 2 Hz and 20 Hz, between 2 Hz and 15 Hz, etc.) and a higher frequency range (e.g., centered around 30 Hz, e.g., between 20 Hz and 50 Hz, between 22 Hz and 45 Hz, between 22 Hz and 40 Hz, between 25 Hz and 35 Hz, etc.). The lower frequency range may be stimulated for between about 10 seconds and 2 minutes (e.g., between about 10 seconds and 2 minutes, between about 20 seconds and 90 minutes, between about 30 second and 90 seconds, etc.). The higher frequency range may be applied for a shorter duration, e.g., between 0.5 seconds and 10 seconds, between 1 second and 5 seconds, between 1 second and 3 seconds, etc.). The higher frequency range may be paired with a motor task.

For example, described herein are methods and apparatuses configured for performing these methods that includes the use of two frequency ranges, a low range and a high range, wherein the higher range is paired with learning and/or performance of a motor task. The low range and high range frequency stimulation may be applied to a vagus nerve pathway (e.g., the vagus nerve or nerves that activate the vagus nerve). For example, these methods may include applying vagus nerve stimulation centered around about 10 Hz for about 60 seconds in duration, and also applying vagus nerve stimulation at a frequency centered around 30 Hz while paired with a motor learning skill (which may be focused on improving motor-pathways by temporally pairing VNS to motor tasks and centered around 30 Hz stimulation over a few seconds). The low frequency range stimulation may be applied to the same nerve or a different nerve as the higher frequency range stimulation. For example, the low frequency range stimulation may be applied to one or more of: a vagus nerve, a trigeminal nerve, a splenic nerve, an auricular nerve, or a sacral nerve. The high-frequency range stimulation may be applied to one or more of: a vagus nerve, a trigeminal nerve, a splenic nerve, an auricular nerve, or a sacral nerve.

For example, described herein are methods of treating a neurodegenerative disorder, comprising applying a first nerve stimulation at a first low frequency of less than 20 Hz for a first time period having a first duration of between 10 seconds and 2 minutes and applying a second nerve stimulation at a frequency of greater than 20 Hz for a second time period that is less than the first time period, wherein the first vagus nerve stimulation at least partially overlaps with the second nerve stimulation. The first and/or second nerve stimulation may comprise vagus nerve stimulation. In general, the total charge per day delivered by the first and second nerve stimulation is between about 2.5 nC to about 7.5 mC per day. Any of these methods may include pairing the second nerve stimulation with performance of a motor task.

The first and second nerve stimulation comprises vagus and/or splenic nerve stimulation. The second time period may be between about 0.5 seconds and about 10 seconds. Applying the first and second nerve stimulation comprises applying for less than about 10 minute each day. Applying the first nerve stimulation may comprise applying stimulation to the vagus nerve from an implanted neurostimulator attached or adjacent to the vagus nerve.

The methods may include adjusting the applied nerve stimulation based on the level of a marker, which may include detecting a marker for demyelination in a blood, sputum, or cerebrospinal fluid sample.

For example, a method of reducing demyelination and/or increasing remyelination may include applying a first nerve stimulation at a first low frequency of less than 20 Hz for a first time period having a first duration of between 10 seconds and 2 minutes and applying a second nerve stimulation at a frequency of greater than 20 Hz for a second time period that is less than the first time period, further comprising pairing the application of the second nerve stimulation with performance of a motor task, wherein the first and second nerve stimulation to the patient delivers between 2.5 nC to 7.5 mC per day. The application of the first nerve stimulation may at least partially overlaps with or may be adjacent (e.g., immediately adjacent, e.g., within a about 10 minutes, 7 minutes, 5 minutes, 3 minutes, 1 minute, 45 seconds, 30 seconds, 15 seconds, etc.) to the application of the second nerve stimulation.

The application of the nerve stimulation may comprise applying stimulation to a vagus nerve and/or a splenic nerve. Applying the second nerve stimulation may comprise applying between for between 1 and 15 seconds.

Also described herein are methods and/or software for performing any of these methods. For example, a system for treating a patient may include: one or more electrodes; one or more pulse generator; one or more processors; and a memory coupled to the one or more processors, the memory storing computer-program instructions, that, when executed by the one or more processors, perform a computer-implemented method comprising: applying a first nerve stimulation at a first low frequency of less than 20 Hz for a first time period having a first duration of between 10 seconds and 2 minutes and applying a second nerve stimulation at a frequency of greater than 20 Hz for a second time period that is less than the first time period, wherein the first and second nerve stimulation to the patient delivers between 2.5 nC to 7.5 mC per day.

The process may be further configured to perform a method comprising guiding a user to perform a motor task during the application of the second nerve stimulation. This may include oral/verbal and/or visual guidance.

Also described herein are methods of modulating gene expression of inflammatory cytokines in the CNS and/or neurotransmitter pathway genes, the method comprising applying vagus nerve stimulation to the patient of between 2.5 nC to 7.5 mC per day. Applying may comprise applying the vagus nerve stimulation at between 0.1 and 20 Hz to the vagus nerve. Applying may comprises applying the vagus nerve stimulation for less than 10 minute each day. Applying may comprise applying stimulation to the vagus nerve from an implanted neurostimulator attached or adjacent to the vagus nerve.

Any of these methods may include adjusting the applied vagus nerve stimulation based on the level of a marker. The method may include detecting a marker for demyelination in a blood, sputum, or cerebrospinal fluid sample.

Also described herein are methods of reducing demyelination and/or increasing remyelination, the method comprising pairing the application of a nerve stimulation to the patient of between 2.5 nC to 7.5 mC per day while performing a motor task. The application of the nerve stimulation may comprise applying stimulation to a vagus nerve or a splenic nerve. The application of the nerve stimulation comprises applying between stimulation at a frequency of greater than 20 Hz. The application of the nerve stimulation may comprise applying between stimulation at a frequency of greater than 30 Hz. The application of the nerve stimulation may comprise applying between for between 1 and 15 seconds.

All of the methods and apparatuses described herein, in any combination, are herein contemplated and can be used to achieve the benefits as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1H illustrate the use of a treatment regimen to stimulate a nerve to reduce infiltration of inflammatory cells in the central nervous system (CNS).

FIGS. 2A-2C illustrate the use of a treatment regimen to stimulate a nerve to reduce entry of pathogenic cells into the CNS during peak EAE.

FIGS. 3A-3H illustrate the use of a treatment regimen to stimulate a nerve to maintain the integrity of tight junction proteins and to restrict fibrinogen leakage in the parenchyma.

FIG. 4 is a graph showing the use of a modified form of vagus nerve stimulation to modulate gene expression of inflammatory cytokines in the CNS.

FIG. 5 is a graph showing the use of a modified form of vagus nerve stimulation to modulate neurotransmitter pathway genes in the CNS.

### DETAILED DESCRIPTION

Diseases (e.g., diseases and disorder of myelination) which may benefit from neural stimulation, including but not limited to VNS, as described herein (e.g., the methods and apparatuses described herein) include, but are not limited to, multiple sclerosis (MS), Alzheimer's disease, Parkinson's disease, Huntington's disease, Amyotrophic lateral sclerosis (ALS), chronic inflammatory demyelinating polyneuropathy (CIDP), and Batten disease. Other neuroinflammatory disorders may include: acute disseminated encephalomyelitis (ADEM), acute optic neuritis (AON), transverse myelitis, and Neuromyelitis optica (NMO). Neuropathies that may benefit from VNS include peripheral neuropathies, cranial neuropathies, and autonomic neuropathies. Thus any of the methods and apparatuses described herein may be used (and adapted for) treatment with any of these diseases and neuropathies.

The apparatuses (e.g., devices, systems, including software, hardware and firmware) and methods for applying neural stimulation described herein may treat neurodegenerative and neuroinflammatory disorders. Specifically, these apparatuses and methods may reduce demyelination (e.g., by preventing the immune cell infiltration into the CNS) and, surprisingly, may promote remyelination and/or reverse demyelination in order to treat various neurodegenerative and neuroinflammatory disorders such as, but not limited to, multiple sclerosis. As mentioned, other conditions that may be treated may include: ALS, myasthenia gravis, spinal cord injury, stroke, traumatic brain injury, Parkinson's, and epilepsy.

In general, these methods and apparatuses (e.g., systems for performing these methods) may include bimodal stimulation. For example, these methods may include the simultaneous (e.g., overlapping and/or concurrent or sequential) use of electrical stimulation of a target nerve or nerves at two separate frequency ranges, a low frequency range (e.g., centered around 10 Hz, between 1 Hz and 29 Hz, between 2 Hz and 25 Hz, between 2 Hz and 20 Hz, between 2 Hz and 15 Hz, etc.) and a higher frequency range (e.g., centered around 30 Hz, e.g., between 20 Hz and 50 Hz, between 22 Hz and 45 Hz, between 22 Hz and 40 Hz, between 25 Hz and 35 Hz, etc.). The lower frequency range may be stimulated for between about 10 seconds and 2 minutes (e.g., between about 10 seconds and 2 minutes, between about 20 seconds and 90 minutes, between about 30 second and 90 seconds, etc.). The higher frequency range may be applied for a shorter duration, e.g., between 0.5 seconds and 10 seconds, between 1 second and 5 seconds, between 1 second and 3 seconds, etc.). The higher frequency range may be paired with a motor task.

For example, described herein are methods and apparatuses configured for performing these methods that includes the use of two frequency ranges, a low range and a high range, wherein the higher range is paired with learning and/or performance of a motor task. The low range and high range frequency stimulation may be applied to a vagus nerve pathway (e.g., the vagus nerve or nerves that activate the vagus nerve). For example, these methods may include applying vagus nerve stimulation centered around about 10 Hz for about 60 seconds in duration, and also applying vagus nerve stimulation at a frequency centered around 30 Hz while paired with a motor learning skill (which may be focused on improving motor-pathways by temporally pairing VNS to motor tasks and centered around 30 Hz stimulation over a few seconds). The low frequency range stimulation may be applied to the same nerve or a different nerve as the higher frequency range stimulation. For example, the low frequency range stimulation may be applied to one or more of: a vagus nerve, a trigeminal nerve, a splenic nerve, an auricular nerve, or a sacral nerve. The high-frequency range stimulation may be applied to one or more of: a vagus nerve, a trigeminal nerve, a splenic nerve, an auricular nerve, or a sacral nerve.

In some examples a bimodal or multimodal stimulation apparatus (e.g., system) may include a vagus nerve stimulation (VNS) device (or VNS device/splenic device and/or a transcutaneous stimulation device, etc.) that is configured to stimulate both at a low frequency range, e.g., at one frequency of less than 30 Hz (<30 Hz) when not paired to motor training, and may stimulate at the high frequency range, e.g., at a frequency greater than 20 Hz, greater than 25 Hz, 30 Hz or greater, 35 Hz or greater, etc. The apparatus may be configured to provide a paired higher frequency range stimulation and may guide a user to perform a paired motor task. The duration of the paired high frequency range stimulation may be, e.g., up to 10 seconds or more, during which the stimulation and motor task may be done together.

In some cases the neural stimulation may include stimulation of a nerve that activates one or more of: a vagus nerve, a trigeminal nerve, a splenic nerve, an auricular nerve, or a sacral nerve. Activating the nerve (or nerve pathway) in the patient may comprise administering a treatment regimen to a nerve target comprising one or more of these nerves to treat neurodegenerative and neuroinflammatory disorders, and more specifically to reduce demyelination and/or to promote remyelination to treat various neurodegenerative and neuroinflammatory disorders such as multiple sclerosis.

For example, in some cases these methods and apparatuses may include vagus nerve stimulation to treat neurodegenerative and neuroinflammatory disorders, and more specifically to vagus nerve stimulation to reduce demyelination and/or to promote remyelination to treat various neurodegenerative and neuroinflammatory disorders such as multiple sclerosis.

For example, described herein are apparatuses (e.g., devices and/or systems) for reducing demyelination and/or increase remyelination by applying a treatment regimen (e.g., electrical stimulation within a defined parameter set) to a nerve. These apparatuses may be implants or implanted into the patient's body. Any of these apparatuses may include: an optional biosensor configured to detect one or more biomarkers; a stimulator configured to apply stimulation to the vagus nerve; and a controller coupled to the biosensor and the stimulator and configured to apply stimulation to the vagus nerve from the stimulator sufficient to reduce demyelination and/or increase remyelination of nerves within the patient when the biosensor detects a biomarker indicative of demyelination. The controller may, in particular, be configured to apply and coordinate the application of both the low frequency range stimulation and the high frequency range stimulation. In some example the controller may coordinate with a second controller that may apply either the low frequency range stimulation or the high frequency range stimulation (e.g., to a different nerve) while the primary controller applies the other type of stimulation (e.g., high frequency range stimulation or low frequency ranges stimulation). In some variations, these apparatuses include an implant comprising a stimulator (e.g., a waveform and/or pulse generator, an oscillator, a power supply and/or power regulation circuit, etc.), a stimulation applicator (e.g., one or more electrodes, mechanical transducers, etc.), and a controller. In some cases the same stimulator may apply both high and low frequency ranges stimulation; in some case a second stimulator may be included and configured to apply either the high frequency range stimulation or low frequency ranges stimulation. The controller may be configured as a microcontroller and may be in electrical communication with the stimulator so as to control operation of the stimulator. The controller may include one or more processors, a memory and/or a timer. The stimulator and/or controller may be in electrical communication, one or more stimulation applicators. In some variations the controller may include or be in communication with wireless communications circuitry for wirelessly communicating with one or more remote processors. The remote processor may be a hand-held device (e.g., smartphone, wearable electronics, etc.). The controller may optionally be in communication with one or more biosensors that may be included with the implant or may be remote from the implant (e.g., may be wearable, single-use, etc.). In some variations including a biosensor, the biosensors are wirelessly connected to the apparatus.

In some variations the apparatus may be used without a biosensor. For example, the apparatus may be configured to periodically and/or on demand apply treatment (e.g., VNS treatment) to prevent or reduce demyelination. The apparatus may be configured to apply treatment doses once multiple times per day (e.g., 1× day, 2×, day, 3×, day, 4× day, 5× day, 6× day), or every other day, or every 3 days, etc. In some variations the apparatus may be configured to both automatically apply a treatment dose on a predetermined and/or adjustable scheduled, as well as provide treatment doses based on input from a user (e.g., patient, physician, etc., including “on demand” doses) and/or based on detection of a biomarker indicative of an actual or potential increase in demyelination.

In any of these variations, a biosensor may be included and may be configured to detect one or more markers (e.g., biomarkers) from the patient's body, including from the patient's blood and/or cerebrospinal fluid. Examples of biomarkers may be found herein. The biosensor may be part of the implanted apparatus, or it may be connected to the apparatus (e.g., the controller) via a wired or wireless communication. The biosensor may be configured to detect any biological marker, including chemical markers (e.g., a protein, nucleotide, e.g., RNA, DNA, microRNA, etc., lipid, carbohydrate, etc.), as well as functional markers (nerve conduction, etc.), body temperature, and the like. For example, in some variations, the biosensor is configured to detect temperature.

In general, the apparatuses described herein may be configured to be inserted or implanted into the body. For example, the apparatus may be configured to be implanted. The apparatus may include one or more stimulation applicators (also referred to as simply a stimulator or in some variations a VNS treatment stimulator) that may be a mechanical and/or electrical stimulator. A mechanical stimulator may be a piezoelectric driver that may vibrate and/or apply pressure to the tissue, including to the vagus nerve, in the VNS treatment parameters, such as mechanical stimulation of the vagus nerve at between the target frequency range for a treatment time. Alternatively or additionally, the stimulation applicator may be an electrical stimulation applicator and may include one or more (e.g., two or more) electrodes configured to apply electrical stimulation to the target nerve (e.g., vagus nerve). For example, electrical stimulation of about 0.1 mA to 10 mA (e.g., between 1 mA-5 mA), at a frequency of between about 1 Hz and about 2 kHz, depending on the target frequency range being applied (e.g., a low frequency range of less than 30 Hz and a higher frequency range of, e.g., between about 20-100 Hz, etc.), where the pulses applied have a pulse width of between about (50-500 microsecond, e.g., between about 100-300 usec, etc.). In some examples the controller may be configured to enforce an ‘off-time’ following a paired treatment dose of between about 10 minute and 12 hours (e.g., between about 2 hours and 10 hours, between about 3 hours and 6 hours, at least 2 hours, at least 3 hours, at least 4 hours, at least 5 hours, at least 6 hours, etc.). For example, the stimulator may include an electrode configured to apply electrical energy to the vagus nerve.

In some variation the apparatus is configured to apply treatment to the patient in which the treatment (e.g., VNS) is electrical stimulation. For example, the treatment may include the application of electrical energy at between about 1-100 Hz (e.g., depending on the high frequency range stimulation or low frequency range stimulation being applied) between about 1-50 Hz, etc.). The energy may have a peak amplitude of between about 0.1 mA and about 2 mA (e.g., between about 0.2 mA and about 1.8 mA, between about 0.5 mA and about 1.5 mA, between about 0.5 mA and about 1 mA, between about 0.1 mA and about 1 mA, approximately 0.5 mA, approximately 0.75 mA, approximately 1 mA, etc.). Alternatively the applied energy may have an average amplitude of between about 0.1 mA and about 2 mA (e.g., between about 0.2 mA and about 1.8 mA, between about 0.5 mA and about 1.5 mA, between about 0.5 mA and about 1 mA, between about 0.1 mA and about 1 mA, approximately 0.5 mA, approximately 0.75 mA, approximately 1 mA, etc.). The applied energy is typically pulsed, and may be pulsed square waves, sinusoidal waves, triangular waves, etc. The applied energy may be biphasic or monophasic. For example, the applied energy may be biphasic. The applied VNS treatment may be a constant biphasic pulse train having a frequency of between 1-100 Hz (e.g., 10 Hz) and a peak amplitude of between about 0.5 mA and 2 mA (e.g., approximately 0.75 mA). Any of the methods for treatment described herein may be configured to apply this type of VNS treatment.

Any of the apparatuses (e.g., devices, systems, etc.) described herein may be configured to be implanted on the target nerve or nerves, including, e.g., a vagus nerve. Thus, any of these apparatuses may be implanted via a nerve sheath or nerve cuff configured to secure the apparatus onto the nerve and/or prevent movement of the apparatus relative to the nerve and/or insulate the apparatus from other tissues. The implanted apparatus may be implanted in any appropriate location on the nerve, including one or around the vagus nerve at the upper chest, or on or around the vagus nerve at a sub-diaphragmatic location. The implant may be a leadless implant that is connected to the vagus (see, e.g., U.S. Pat. Nos. 8,412,338, 8,612,002, 8,886,339, and 8,788,034, each of which is herein incorporated by reference in its entirety). For example, any of these apparatuses may include a nerve cuff configured to secure the stimulator to the vagus nerve. Alternatively, any of these apparatuses may include a lead connecting the micro stimulator and/or other components to the stimulation applicator on/around the vagus nerve via one or more leads.

As mentioned, any of these apparatuses may be configured to apply VNS treatment comprising a low duty-cycle electrical stimulation of between about 0.25 mA and about 5 mA to the vagus nerve for less than about 2 minutes. The apparatus may be configured to provide an off-time of at least x minutes/hours (e.g., 10 minutes, 20 minutes, 30 minutes, 40 minutes, 60 minutes, 2 hours, 3 hours, 4 hours, etc.).

Any of the apparatuses described herein may be configured to perform a method of reducing demyelination and/or a method of increasing remyelination in a patient diagnosed with or at risk of a disorder involving demyelinated nerves (e.g., including but not limited to methods of treating a disorder and/or disease associated with demyelination, such as multiple sclerosis). For example, a method of reducing demyelination (and/or a method of increasing remyelination) may be a method comprising detecting a marker for demyelination and applying stimulation to the vagus nerve to reduce demyelination of nerves within the patent.

Applying stimulation to the vagus nerve includes applying VNS treatment and may comprise, for example, applying electrical stimulation of between about 0.25 and about 5 mA to the vagus nerve for less than about 2 minutes. In some variations this may include waiting for an off-time (e.g., an off-time of at least 10 minutes, 30 minutes, 1 hour, 2 hours, 3 hours, 4 hours, etc.).

Any of these methods may include applying non-invasive stimulation to the vagus nerve. For example, the simulation may be through a transdermal (e.g., via a surface electrode and/or mechanical stimulation, including ultrasound) route over a portion of the vagus nerve. The vagus nerve includes a number of branches or extensions that may be accessed and/or targeted from outside of the body either mechanically and/or electrically. For example, non-invasive application may include ultrasound stimulation of a target nerve or nerves (e.g., vagus nerve) at the high frequency range stimulation or low frequency range stimulation. Any of these methods may include applying transdermal electrical stimulation (TENS), or the like.

Any of the methods described herein may include monitoring, e.g., periodically, on demand, and/or continuously, one or more markers (e.g., biomarkers) for demyelination or a risk of demyelination. As mentioned, any appropriate method or apparatus for monitoring demyelination or a risk of demyelination may be used. For example any of these methods may include detecting a marker for demyelination comprising monitoring the patient's temperature. A change (including an increase) in core body temperature has been linked to an increase in symptoms in demyelination disorders, including but not limited to MS.

Any of the methods and apparatuses described herein may be used with or linked to markers for the integrity of the blood-brain barrier. The methods and apparatuses described herein generally improve the integrity of the blood-brain barrier. Thus, any marker linked to leakage or loss of integrity of the blood-brain barrier may be used to trigger VNS therapy as described herein. Examples of markers may include Serum S1008, as well as imaging modalities such as contrast-enhanced magnetic resonance imaging, CT-scan and lumbar puncture.

The detection of one or more markers (e.g., biomarkers) for demyelination may include determining a level of tumor necrosis factor in a blood or cerebrospinal fluid sample.

For example, described herein are methods (e.g., methods of treating a demyelination disorder, such as but not limited to MS, and/or methods of reducing or reversing demyelination) that include: detecting demyelination in a patient, and applying stimulation, both high frequency range stimulation or low frequency range stimulation, to the target (e.g., vagus) nerve or nerves to increase the remyelination of nerves within the patent.

For example, any of these methods may include repeatedly applying a low duty-cycle electrical stimulation of between about 0.25 and about 5 mA to the patient's vagus nerve for less than about 2 minutes, followed by an off-time (e.g., of between about 10 minutes and about 48 hours) before the next stimulation at a high frequency range stimulation and at a low frequency range stimulation. The high frequency range stimulation may be paired with a motor task.

Any of these methods and apparatuses may also include or be adapted to include the concurrent (immediately before, during or after, including systemically and/or locally) treatment with one or more pharmacological agents, particularly those that are believed to help with a demyelinating condition, such as (but not limited to) MS. For example, any of these method may include concurrently treating with a pharmacological agent such as one or more of: interferon beta-1a, interferon beta-1b, glatiramer acetate, glatiramer acetate, peginterferon beta-1a, daclizumab, teriflunomide, fingolimod, dimethyl fumarate, alemtuzumab, mitoxantrone, ocrelizumab, natalizumab.

As used herein paired performance of a motor task (also referred to herein as a motor skill) may include any appropriate motor skill or task, including a new (to the patient) motor skill/task. Performance as contemplated herein may refer to an act of executing a motor skill or task. Continuous practice of a specific motor skill can result in improved performance or improved case of performance, which leads to motor learning. Motor learning is a relatively permanent change in the ability to perform a skill as a result of continuous practice or experience. Motor skills are movements and actions of the muscles. There are two major groups of motor skills, gross and fine motor skills. Gross motor skills require the use of large muscle groups in our legs, torso, and arms to perform tasks such as: walking, balancing, and crawling. The skill required is not extensive and therefore are usually associated with continuous tasks. Gross motor skills can be used repeatedly without putting much thought or effort into them. Gross motor skills can be further divided into two subgroups: Locomotor skills, such as running, jumping, sliding, and swimming; and object-control skills such as throwing, catching, dribbling, and kicking. Fine motor skills require the use of smaller muscle groups to perform smaller movements. These muscles include those found in wrists, hands, fingers, feet and in toes or in the case of other mammals in their hooves or paws, etc. These tasks are precise in nature, for example, playing the piano, tying shoelaces, combing hair, brushing teeth, shaving or other fine motor skill. Some fine motor skills may be susceptible to retention loss if not in use, these skills can be lost if not used frequently. Fine motor skills need to continuously be used. Discrete tasks such as switch gears in an automobile, grasping an object, or striking a match, usually require more fine motor skill than gross motor skills. In certain embodiments, gross and/or fine motor skills are included in learned skills contemplated herein alone or in combination with VNS and/or pharmaceutical treatments for a demyelinating condition or disease

As mentioned, any of the methods and apparatuses described herein may include continuously monitoring the patient for demyelination or a condition implicated in demyelination. For example, any of these methods and apparatuses described herein may include monitoring the patient for a marker related to a disease selected from the group consisting of neurodegenerative diseases, neuroinflammatory diseases, and neuropathies. In some examples, the method includes detecting demyelination in a patient by detecting a marker related to MS. For example, the marker (e.g., biomarker) may be selected from the group including: neurofilament, glial fibrillary acidic protein, the monocyte macrophage marker CD163, the glial activation marker YKL-40, the B cell chemoattractant CXCL13, miRNA, mRNA, myelin reactive t cells, Kir4.1 antibodies, osteopontin, and microbiome associated lipopeptides.

In particular, described herein are methods and apparatuses for reducing or preventing demyelination and/or for increasing remyelination by stimulation of a vagus nerve. For example, an apparatus (e.g., a system, device, assembly, etc., including implants), may include: a vagus nerve stimulator configured to be implanted over or adjacent to a vagus nerve; one or more electrodes on the vagus nerve stimulator configured to apply electrical stimulation to the vagus nerve; and a controller coupled to the vagus nerve stimulator and configured to apply electrical stimulation to the vagus nerve from the one or more electrodes, wherein the controller is constrained to apply a charge per day of between 2.5 nC and 7.5 mC to reduce demyelination and/or increase remyelination within the patient. This apparatus may be a system.

The system may include an input configured to receive one or more marker level indicators, wherein the controller is configured to adjust the applied charge based on the one or more marker level indicators. For example, the system may include a biosensor configured to detect the marker from the patient's blood and/or cerebrospinal fluid and to determine a maker level indicator.

The controller may be configured to deliver the electrical stimulation during one or more dose sessions of about 5 minutes or less (e.g., 4 min or less, 3 min or less, 2 min or less, 1 min or less, etc.). The controller may be configured to apply the charge per day at a frequency of between 1 and 20 Hz. In some variations the controller is configured to apply the charge per day at a frequency of between 1 and 12 Hz.

In any of these apparatuses, the system is configured to be implanted.

Any of these systems may include a nerve cuff configured to secure the vagus nerve stimulator to the vagus nerve. The controller may be configured to apply the charge per day at two distinct frequencies between 1 and 20 Hz. The controller may be configured to apply a first dose of the electrical stimulation to reduce demyelination at a first frequency between 1 and 20 Hz, and a second dose of electrical stimulation to increase remyelination within the patient at a second frequency that is higher than the first frequency. For example, the first dose of electrical stimulation may have a frequency less than 10 Hz, and the second dose of electrical stimulation has a frequency ranging from 10 Hz and 50 Hz (e.g., 15 Hz to 45 Hz, etc.). In some variations the first dose of electrical stimulation has a frequency of ranging from 1 Hz and 10 Hz, and the second dose of electrical stimulation has a frequency ranging from 10 Hz and 50 Hz. In any of these examples, the low frequency range stimulation and the high frequency range stimulation may be separated for a particular treatment; for example, the frequency of the low frequency stimulation may be separated from the frequency of the high frequency stimulation by at least 5 Hz.

Also described herein are method of increasing clearance of myelin debris in a patient diagnosed with or at risk of a disorder involving demyelinated nerves, the method comprising applying vagus nerve stimulation to the patient of between 2.5 nC to 7.5 mC per day. The Applying may comprise applying the vagus nerve stimulation at between 0.1 and 20 Hz to the vagus nerve and applying a second frequency of >20 Hz for a different (and potentially overlapping time period). In some variations, applying comprises applying a the vagus nerve stimulation for less than about 5 minute each day (e.g., less than about 4 min per day, less than about 3 min per day, less than about 2 min per day, less than about 1 min per day, etc.). Applying may comprise applying stimulation to the vagus nerve from an implanted neurostimulator attached or adjacent to the vagus nerve.

Any of these methods may include adjusting the applied vagus nerve stimulation based on the level of a marker. For example, the method may include detecting a marker for demyelination in a blood, sputum, and/or cerebrospinal fluid sample.

A system for reducing demyelination and/or increasing remyelination by stimulation of a vagus nerve may include: a vagus nerve stimulator configured to be implanted over or adjacent to a vagus nerve; one or more electrodes on the vagus nerve stimulator configured to apply electrical stimulation to the vagus nerve; and a controller coupled to the vagus nerve stimulator and configured to apply electrical stimulation to the vagus nerve from the one or more electrodes, wherein the controller is constrained to apply a low duty-cycle electrical stimulation for a duration of between 1 second and 5 minutes per day, the electrical stimulation comprising a first dose of electrical stimulation to reduce demyelination at a first frequency of between 1 and 20 Hz, and a second dose of electrical stimulation to increase remyelination at a second frequency that is higher than the first frequency.

The controller may be configured to apply electrical stimulation between 1 and 24 times per day. The frequency of the first dose of electrical stimulation may be between 1 Hz to 10 Hz. The frequency of the first dose of electrical stimulation may be between 1 Hz to 5 Hz. The frequency of the second dose of electrical stimulation may be between 10 Hz to 30 Hz. The first dose of electrical stimulation may have a frequency less than 5 Hz, and the second dose of electrical stimulation may have a frequency ranging from 10 Hz and 30 Hz.

As mentioned, the controller may be configured to modulate the electrical stimulation based on feedback from a user or based on one or more biomarkers associated with demyelination. The controller may be configured to reduce the frequency of the electrical stimulation based on the feedback. For example, a controller may be configured to adjust the duration of the first dose and the second dose based on the feedback.

A method of reducing demyelination and/or increasing remyelination in a patient having a disorder involving demyelinated nerves may include: applying a low duty-cycle electrical stimulation for a total duration of between 1 second and 5 minutes per day, the electrical stimulation comprising a first dose of electrical stimulation to reduce demyelination at a first frequency of between 1 and 20 Hz, and a second dose of electrical stimulation to increase remyelination at a second frequency that is higher than the first frequency. The low duty-cycle electrical stimulation may be applied, e.g., from 1 to 24 times per day.

The first frequency may range from 1 Hz to 10 Hz. In some variations, the first frequency ranges from 1 Hz to 5 Hz. The second frequency may range from 10 Hz to 30 Hz. The first dose of electrical stimulation may have a frequency less than 5 Hz, and the second dose of electrical stimulation may have a frequency ranging from 10 Hz and 30 Hz.

Any of these method may also include modulating the electrical stimulation based on feedback from a user or based on one or more biomarkers associated with demyelination. The controller may be configured to adjust the duration of the first dose and the second dose based on the feedback.

As described herein, any of these methods may include concurrently administering one or more of an Interferon β drug, glatiramer acetate, and daclizumab in combination with applying the low duty-cycle electrical stimulation to target interferon β-1a and 1b receptors and T-cell activation to reduce central nervous system inflammation and demyelination. For example, any of these methods may include administering one or more of fingolimod, teriflunomide, and dimethyl fumarate in combination with applying the low duty-cycle electrical stimulation to target lymphocyte migration or activation to reduce central nervous system inflammation and demyelination. Any of these methods may include administering one or more of mitoxantrone, alemtuzumab, ocrelizumab, and natalizumab in combination with applying the low duty-cycle electrical stimulation to induce DNA breakage, CD52 to induce cell lysis, B-cell CD20 antigen for depletion, and/or integrin receptors to alter leukocyte migration, to reduce central nervous system inflammation and demyelination. In some variations, these methods may include administering one or more of clemastine, a Selective Estrogen Receptor Modulator (SERM), and other drugs targeting oligodentrocyte progenitor cells to enhance maturation into myelin-producing oligodendrocytes to enhance remyelination and clinical recovery from central nervous system damage.

Previous therapies using electrical stimulation of nerves to treat demyelination and/or promote remyelination primarily focused on non-motor pathway endpoints, and was centered around about 10 Hz and about 60 seconds in duration. Also described herein are methods and apparatuses that may include any of those described above paired with one or more motor tasks; in particular those in which the applied stimulation (including, but not limited to VNS), improving motor-pathways by temporally pairing VNS to motor tasks and may be centered around 30 Hz stimulation over a few seconds (e.g., between about 0.1 to 5 second, between 0.1 to 3 seconds, between about 0.1 to 2 second, etc.).

Thus, described herein is bimodal or multimodal stimulation, in which the stimulation may include electrical (e.g., VNS, splenic, etc.) stimulation by a device, which may be an implanted device (or a VNS device/splenic device) and/or a transcutaneous device that may be configured to stimulate both at one frequency (e.g., <30 Hz) when not paired with a second stimulation or motor task, and may be driven at a different frequency (e.g., >20 Hz, >25 Hz, >30 Hz, etc.) when paired with a motor training task. For example, the method and apparatuses described herein may including pairing electrical stimulation at greater than 20 Hz for up to 10 seconds with a motor task in some cases and unpaired (e.g., <30 Hz) electrical stimulation in other cases. This combination may be highly effective when done together.

These methods may be used to treat a neurodegenerative disorder, such as, but not limited to Multiple sclerosis (MS), ALS, myasthenia gravis etc., including in particular treating pathways of neurodegenerative disorders that are not inflammatory in nature; specifically treating myelination and neurodegeneration diseases and movement disorders. For example, these methods and apparatuses may be used to treat spinal cord injury, stroke, traumatic brain injury, Parkinson's, epilepsy and others, even where there is not a significant inflammatory component. Any of these methods and apparatuses may also include pairing to motor training for effecting multiple different aspects of therapy.

### EXAMPLES

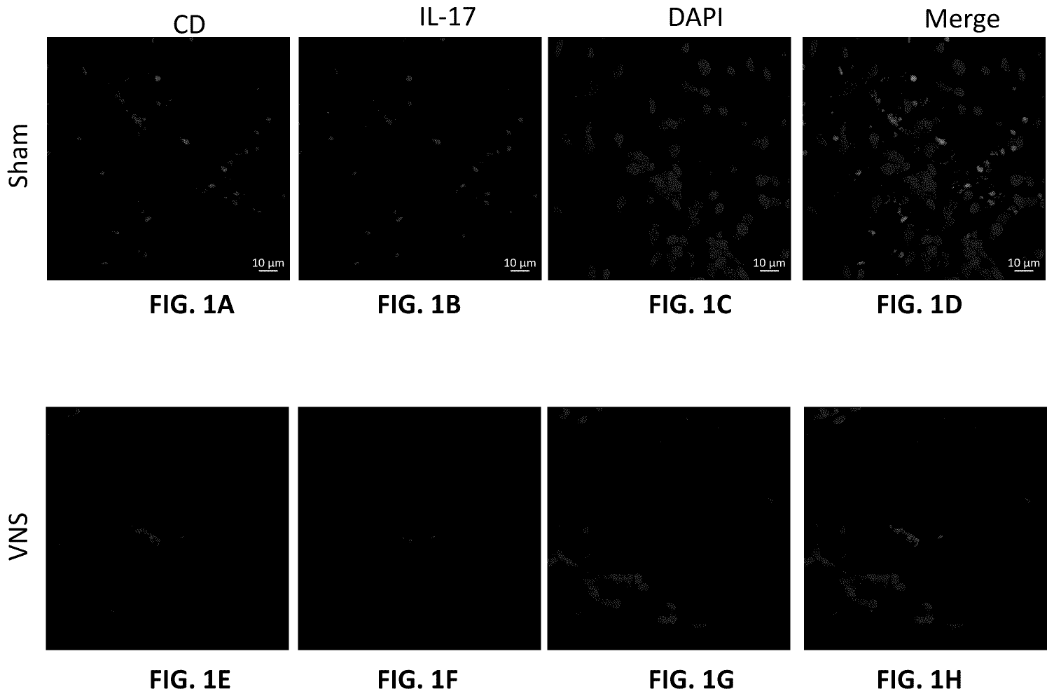

For example, FIGS. 1A-1H illustrate the reduction in infiltrating inflammatory cells in the CNS following the application of energy (electrical stimulation) as described herein. FIGS. 1A-1D show treatment with sham, while FIGS. 1E-1H show the effect of vagus nerve stimulation (VNS). Experimental autoimmune encephalomyelitis (EAE), a well-recognized model for multiple sclerosis (MS) in humans, was induced in rats, and Sham and VNS was performed, and the rats were euthanized at the peak of the disease (days 3-4 post-symptomatic). Lumbar spinal cords were collected, and immunohistochemistry (immunofluorescence) was performed to identify inflammatory T cells.

As shown in FIGS. 1E-1H, the applied electrical stimulation restricted the infiltration of pathogenic CD4+(FIG. 1E, as compared to FIG. 1A, control) IL-17 (FIG. 1F, as compared to FIG. 1B, control), and T-cells (FIG. 1G, as compared to FIG. 1C, control) into the CNS, as more CD4+/IL-17+ T-cells are found in the spinal cords of sham stimulated animals than in the stimulated animals.

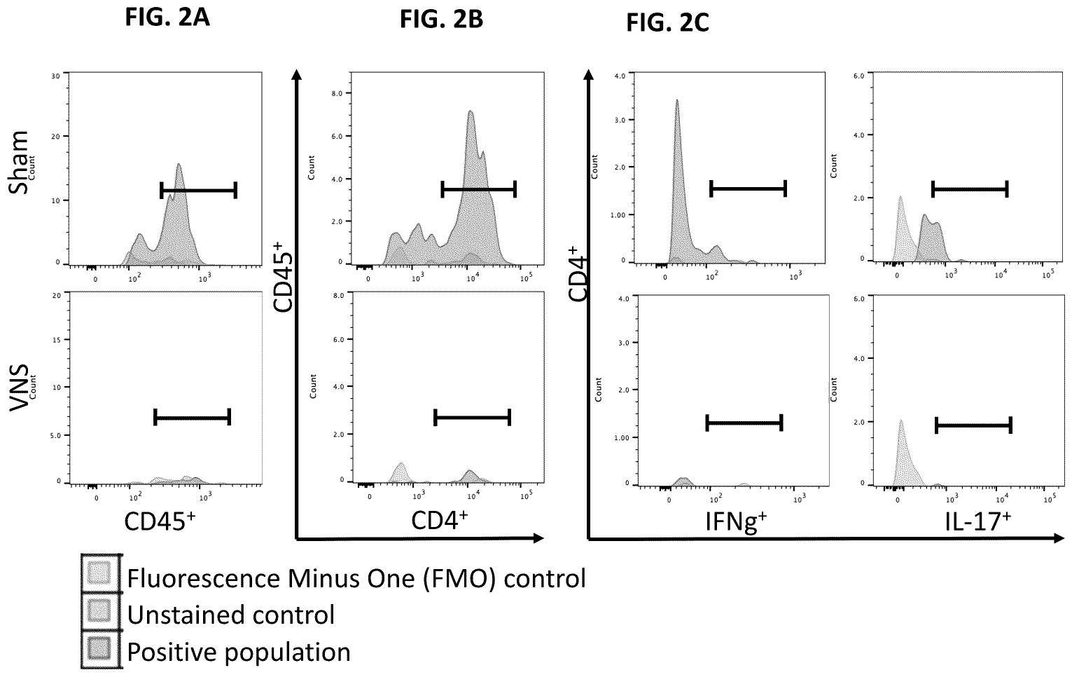

FIGS. 2A-2C illustrate the reduction in entry of pathogenic cells in the CNS during peak EAE in treated vs. control animals. The application of electrical energy significantly reduces the entry of pathogenic cells in the CNS during peak EAE. Spinal cords were collected from Sham and VNS EAE rats at the peak of the disease (days 3-4 post-symptomatic), and flow cytometry was performed. As shown in FIGS. 2A-2C, VNS reduced the infiltration of pathogenic CD4+/IFNg+ and CD4+/IL-17+ T-cells into the CNS as more CD4+/IL-17+ T-cells are found in the spinal cords of sham stimulated animals than in the stimulated animals.

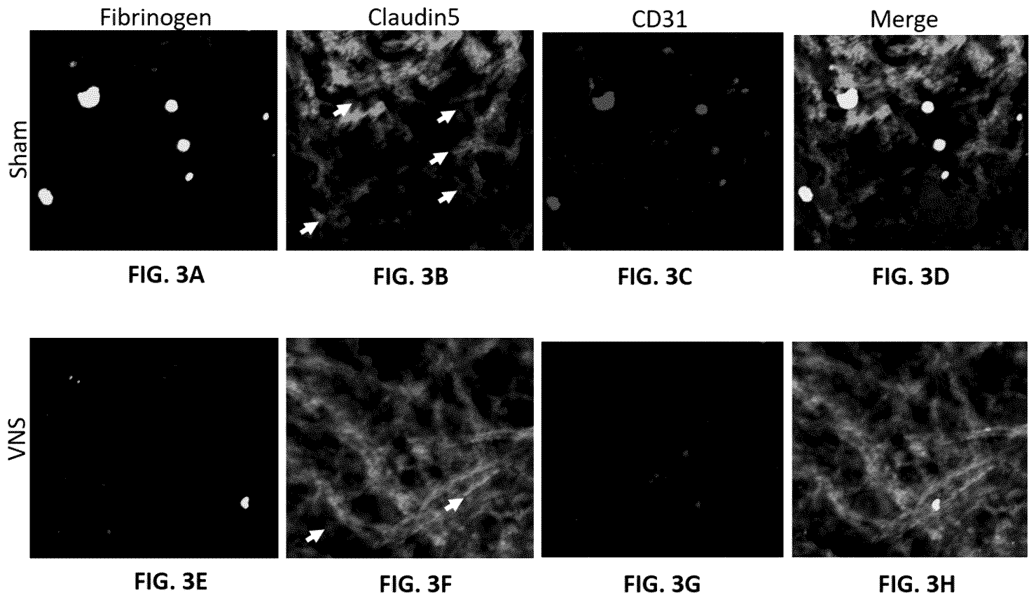

Electrical stimulation (e.g., of the vagus nerve) also helped maintain the integrity of tight junction proteins and restricts fibrinogen leakage in the parenchyma in the neurodegenerative animal model, as shown in FIGS. 3A-3D.

In FIGS. 3A-3H, the application of electrical energy to the vagus nerve maintained the integrity of tight junction proteins and restricted fibrinogen leakage in the parenchyma (compare VNS, FIGS. 3E-3H, to sham control, shown in FIGS. 3A-3D). To evaluate vascular leakiness, immunofluorescence was performed to identify Claudin 5 (tight junction protein, FIG. 3F compared to control FIG. 3B), CD31 (blood vessel marker, FIG. 3G compared to FIG. 3C), and fibrinogen (FIG. 3E compared to FIG. 3A) in Sham and VNS EAE rat lumbar spinal cords, at peak of the disease (days 3-4 post-symptomatic). Arrows in FIGS. 3F and 3B indicate areas of claudin 5 disruption.

As shown, electrical stimulation (e.g., VNS) maintained the integrity of Claudin 5, the most enriched tight junction protein and prevented fibrinogen leakiness in the CNS, as more disruption of claudin 5 along with more fibrinogen deposition was observed in the spinal cords of sham stimulated animals than in the stimulated animals. Electrical stimulation (e.g., VNS) also modulated gene expressions of inflammatory cytokines in the CNS.

### Electrical Stimulation Modulates Gene Expression of Inflammatory Cytokines in the CNS

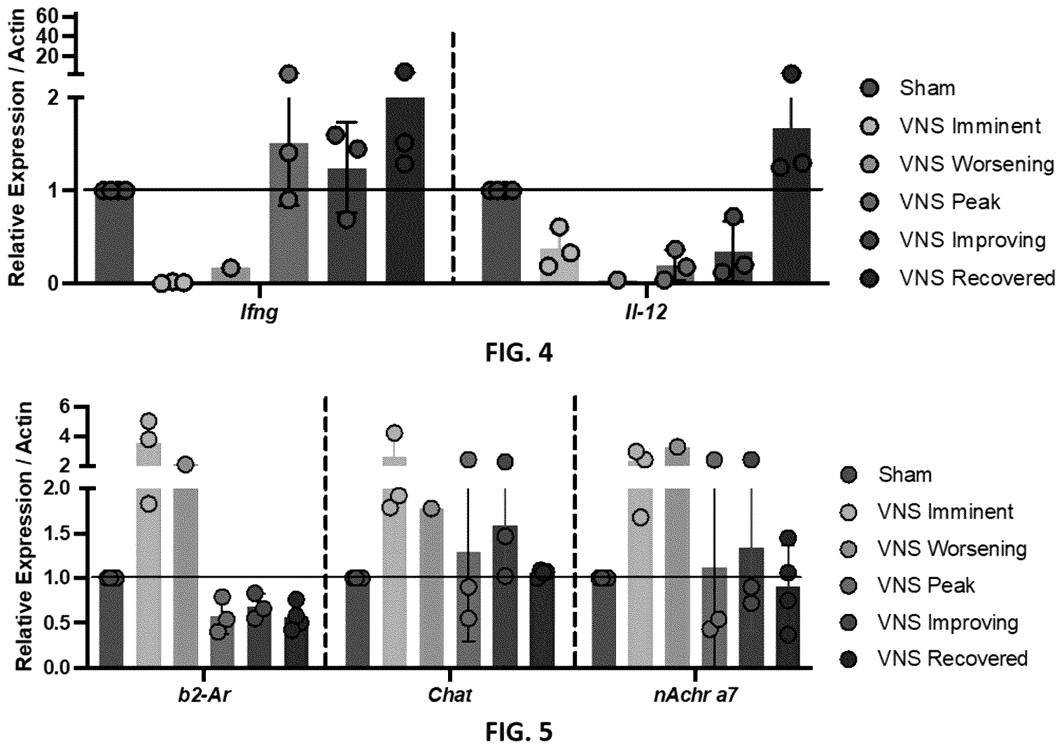

The use of electrical simulation was effective in modulating gene expression of inflammatory cytokines. qPCR was performed in sham and VNS EAE rat lumbar spinal cords to quantitate genes of inflammatory cytokines at different stages of the disease. The five analyzed stages included stages corresponding to when symptomatic disease is imminent (expected day prior to first symptom), worsening (1-2 days post-symptom), peak disease (3-4 days post-symptom), improving disease (5-7 days post symptom) and symptomatically recovered (˜11 days post first symptom). See FIGS. 4 and 5.

As shown in FIG. 4, VNS strongly suppressed the inflammatory Ifng gene expression during the initial stages of the EAE disease compared to sham rats. In the later stages of the disease model, when IFN-g is protective, the Ifng was not suppressed. VNS mediated suppression of Il-12 gene expression throughout the course of symptomatic disease.

Electrical stimulation (e.g., VNS) was also found to modulate neurotransmitter pathway genes in the CNS. For example, qPCR was performed in sham and VNS EAE rat lumbar spinal cords to quantitate gene expression of proteins that are important to the cholinergic anti-inflammatory pathway, at different stages of the disease. The 5 analyzed stages are when symptomatic disease is imminent (expected day prior to first symptom), worsening (1-2 days post-symptom), peak disease (3-4 days post-symptom), improving disease (5-7 days post symptom) and symptomatically recovered (˜11 days post first symptom).

As shown in FIG. 5, electrical stimulation (e.g., VNS) modulates neurotransmitter pathway genes by upregulating beta 2 adrenergic receptor, choline acetyltransferase, and the a7 nicotinic acetylcholine receptor at the early stages of the diseases as compared to shams.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of treating a neurodegenerative disorder, the method comprising applying a first nerve stimulation at a first low frequency of less than 20 Hz for a first time period having a first duration of between 10 seconds and 2 minutes and applying a second nerve stimulation at a frequency of greater than 20 Hz for a second time period that is less than the first time period, wherein the first vagus nerve stimulation at least partially overlaps with the second nerve stimulation.

2. The method of claim 1, wherein the first nerve stimulation comprises vagus nerve stimulation.

3. The method of claim 1, wherein the total charge per day delivered by the first and second nerve stimulation is between 2.5 nC to 7.5 mC per day.

4. The method of claim 1, further comprising pairing the second nerve stimulation with performance of a motor task.

5. The method of claim 1, wherein the first and second nerve stimulation comprises vagus and/or splenic nerve stimulation.

6. The method of claim 1, wherein the second time period is between 0.5 seconds and 10 seconds.

7. The method of claim 1, wherein applying the first and second nerve stimulation comprises applying for less than 10 minute each day.

8. The method of claim 1, wherein applying the first nerve stimulation comprises applying stimulation to the vagus nerve from an implanted neurostimulator attached or adjacent to the vagus nerve.

9. The method of claim 1, further comprising adjusting the applied nerve stimulation based on the level of a marker.

10. The method of claim 9, further comprising detecting a marker for demyelination in a blood, sputum, or cerebrospinal fluid sample.

11. A method of reducing demyelination and/or increasing remyelination, the method comprising applying a first nerve stimulation at a first low frequency of less than 20 Hz for a first time period having a first duration of between 10 seconds and 2 minutes and applying a second nerve stimulation at a frequency of greater than 20 Hz for a second time period that is less than the first time period, further comprising pairing the application of the second nerve stimulation with performance of a motor task, wherein the first and second nerve stimulation to the patient delivers between 2.5 nC to 7.5 mC per day.

12. The method of claim 11, wherein the application of the first nerve stimulation at least partially overlaps with or is adjacent to the application of the second nerve stimulation.

13. The method of claim 11, wherein the application of the nerve stimulation comprises applying stimulation to a vagus nerve and/or a splenic nerve.

14. The method of claim 11, wherein applying the second nerve stimulation comprises applying between for between 1 and 15 seconds.

15. A system for treating a patient, the system comprising:
one or more electrodes;
a pulse generator;
one or more processors; and
a memory coupled to the one or more processors, the memory storing computer-program instructions, that, when executed by the one or more processors, perform a computer-implemented method comprising:
applying a first nerve stimulation at a first low frequency of less than 20 Hz for a first time period having a first duration of between 10 seconds and 2 minutes and applying a second nerve stimulation at a frequency of greater than 20 Hz for a second time period that is less than the first time period, wherein the first and second nerve stimulation to the patient delivers between 2.5 nC to 7.5 mC per day.

16. The system of claim 15, wherein the process is further configured to perform a method comprising guiding a user to perform a motor task during the application of the second nerve stimulation.

17. A method of modulating gene expression of inflammatory cytokines in the CNS and/or neurotransmitter pathway genes, the method comprising applying vagus nerve stimulation to the patient of between 2.5 nC to 7.5 mC per day.

18. The method of claim 17, wherein applying comprises applying the vagus nerve stimulation at between 0.1 and 20 Hz to the vagus nerve.

19. The method of claim 17, wherein applying comprises applying the vagus nerve stimulation for less than 10 minute each day.

20. The method of claim 17, wherein applying comprises applying stimulation to the vagus nerve from an implanted neurostimulator attached or adjacent to the vagus nerve.

21. The method of claim 17, further comprising adjusting the applied vagus nerve stimulation based on the level of a marker.

22. The method of claim 21, further comprising detecting a marker for demyelination in a blood, sputum, or cerebrospinal fluid sample.

23. A method of reducing demyelination and/or increasing remyelination, the method comprising pairing the application of a nerve stimulation to the patient of between 2.5 nC to 7.5 mC per day while performing a motor task.

24. The method of claim 23, wherein the application of the nerve stimulation comprises applying stimulation to a vagus nerve or a splenic nerve.

25. The method of claim 23, wherein the application of the nerve stimulation comprises applying between stimulation at a frequency of greater than 20 Hz.

26. The method of claim 23, wherein the application of the nerve stimulation comprises applying between stimulation at a frequency of greater than 30 Hz.

27. The method of claim 23, wherein the application of the nerve stimulation comprises applying between for between 1 and 15 seconds.
